(19) United States
(12) Patent Application Publication
(10) Pub. No.: US 2026/0130598 A1
(43) Pub. Date: May 14, 2026

(54) BIOLOGICAL INFORMATION PROCESSING DEVICE, BIOLOGICAL INFORMATION PROCESSING METHOD, AND STORAGE MEDIUM

(71) Applicant: TERUMO KABUSHIKI KAISHA, Tokyo (JP)

(72) Inventors: Tomonori HATTA, Hadano Kanagawa (JP); Takayuki UCHIDA, Hiratsuka Kanagawa (JP); Yoko MURAYAMA, Sagamihara Kanagawa (JP)

(21) Appl. No.: 19/444,099

(22) Filed: Jan. 8, 2026

Related U.S. Application Data
(63) Continuation of application No. PCT/JP2024/024634, filed on Jul. 8, 2024.

(30) Foreign Application Priority Data
Jul. 10, 2023 (JP) 2023-113259

(51) Int. Cl. A61B 5/021; A61B 5/00; A61B 5/352; A61B 7/04

(52) U.S. Cl. CPC A61B 5/02116; A61B 5/352; A61B 5/7239; A61B 5/725; A61B 5/7275; A61B 7/04; A61B 2562/0219

(57) ABSTRACT

A biological information processing device acquires biological information including a volume pulse wave and a heart sound, performs second-order differentiation on the volume pulse wave to derive an acceleration pulse wave, determines in the acceleration pulse wave an offset position occurring an offset time after the beginning of a grade II sound, backtracks from a local minimum point subsequent to the offset position to find a first local maximum point, determines a point in the volume pulse wave corresponding to the first local maximum point to be a dicrotic notch, and stores information indicating the dicrotic notch in association with or as part of the biological information.

### CROSS-REFERENCE TO RELATED APPLICATION

This application is a continuation of International Patent Application No. PCT/JP2024/024634 filed Jul. 8, 2024, which is based upon and claims the benefit of priority from Japanese Patent Application No. 2023-113259, filed Jul. 10, 2023, the entire contents of which are incorporated herein by reference.

### BACKGROUND

### Technical Field

The present disclosure relates to a biological information processing device, a biological information processing method, and a non-transitory computer-readable storage medium.

### Related Art

There are conventional methods for detecting a dicrotic notch of a pulse wave. The dicrotic notch is an oscillation caused by aortic valve closure. Since the pulse wave is delimited by the dicrotic notch into a systolic phase and a diastolic phase, detecting the dicrotic notch is widely used for diagnosing the circulatory system such as for calculating blood pressure.

### SUMMARY

With such conventional methods, the accuracy of detecting the dicrotic notch of a pulse wave is not sufficient. Additionally, with the conventional methods, since the accuracy of detecting the dicrotic notch of a pulse wave is not sufficient, there is a challenge regarding the accuracy of calculating a blood pressure value or performing other diagnoses based on the temporal positional relationship between the dicrotic notch and other feature points on the waveform of the pulse wave.

Embodiments of the present disclosure improve accuracy of detecting a dicrotic notch of a pulse wave.

Some aspects of the present disclosure are described below.

A biological information processing device comprises: an interface connectable to a measurement device for measuring biological information including a volume pulse wave and a heart sound; a memory; and a processor configured to execute a program stored in the memory to: acquire the biological information, perform second-order differentiation on the volume pulse wave included in the biological information and derive an acceleration pulse wave from the volume pulse wave, determine, in the acceleration pulse wave, an offset position occurring an offset time after the beginning of a grade II sound included in the heart sound, backtrack from a local minimum point that is subsequent to the offset position to find a first local maximum point, determine a point in the volume pulse wave corresponding to the first local maximum point to be a dicrotic notch of the volume pulse wave, and store information indicating the dicrotic notch in association with or as part of the biological information.

According to embodiments of the present disclosure, the accuracy of detecting the dicrotic notch of the pulse wave is improved.

### BRIEF DESCRIPTION OF DRAWINGS

FIG. 1 is a diagram illustrating a configuration of an information processing system according to an embodiment.

FIG. 2 is a diagram illustrating examples of a volume pulse wave waveform, a velocity pulse wave waveform, and an acceleration pulse wave waveform.

FIG. 3 is a diagram illustrating examples of a volume pulse wave waveform, a velocity pulse wave waveform, and an acceleration pulse wave waveform.

FIG. 4 is a diagram illustrating an example of a search of an acceleration pulse wave.

FIG. 5 is a diagram illustrating another example of a search of an acceleration pulse wave.

FIG. 6 is a block diagram illustrating a configuration of an information processing device of the information processing system.

FIG. 7 is a flowchart illustrating operation of the information processing device.

FIG. 8 is a diagram illustrating examples of a detection result and a calculation result, which correspond to FIG. 7.

FIG. 9 is a diagram illustrating an example of a screen that displays information output from the information processing device.

### DETAILED DESCRIPTION

Hereinafter, embodiments of the present disclosure will be described with reference to the drawings.

In the drawings, the same or corresponding parts are denoted by the same reference numerals. The descriptions of the same or corresponding parts will be omitted or simplified as appropriate.

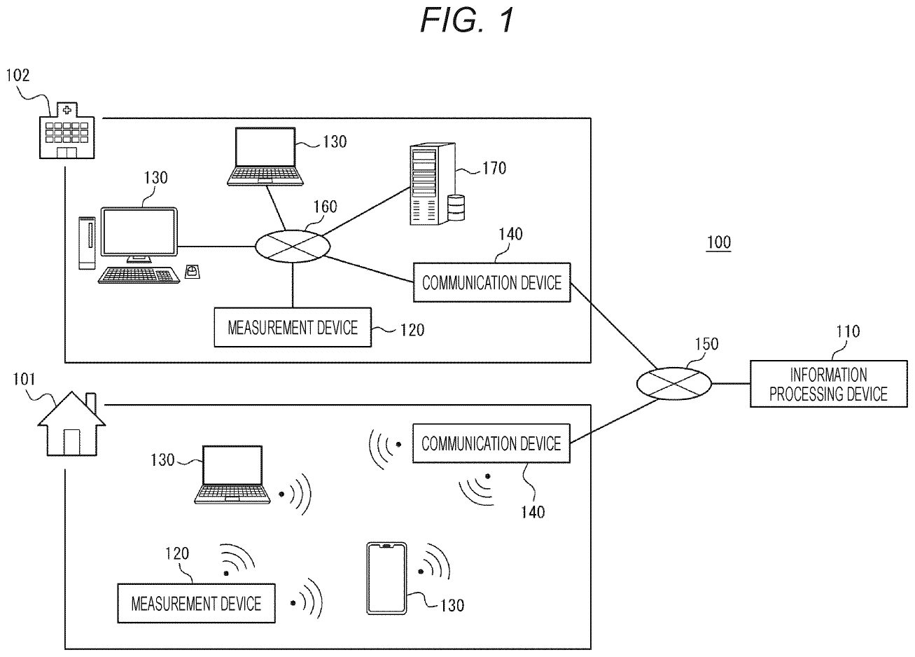

A configuration of an information processing system 100 according to an embodiment will be described with reference to FIG. 1.

The information processing system 100 includes an information processing device 110 and one or more measurement devices 120.

The information processing device 110 is a device that acquires biological information of a subject from the measurement devices 120, performs pulse wave analysis processing, and estimates a value or waveform of intracardiac pressure based on a result of the pulse wave analysis processing. The biological information includes at least a pulse wave, further includes a heart sound in the present embodiment, and preferably further includes an electrocardiogram. The pulse wave included in the biological information is a volume pulse wave in the present embodiment, but may be either the volume pulse wave or a pressure pulse wave. The intracardiac pressure is, for example, right atrium pressure, right ventricular pressure, pulmonary artery pressure, left atrium pressure, left ventricular pressure, femoral artery pressure, central venous pressure, or pulmonary wedge pressure. Since intracardiac pressure periodically fluctuates with each heartbeat, intracardiac pressure may include systolic pressure, diastolic pressure, or mean pressure. For example, right atrium pressure may include systolic pressure, diastolic pressure, or mean pressure. Right atrium pressure is also abbreviated as RAP. Right ventricular pressure may include systolic pressure, diastolic pressure, or end-diastolic pressure. Right ventricular pressure is also abbreviated as RVP. Pulmonary artery pressure may include systolic pressure, diastolic pressure, mean pressure, or end-diastolic pressure. Pulmonary artery pressure is also abbreviated as PAP. Pulmonary artery diastolic pressure is also abbreviated as PADP. Pulmonary artery end-diastolic pressure is also abbreviated as PAEDP. Left atrium pressure may include systolic pressure, diastolic pressure, or mean pressure. Left atrium pressure is also abbreviated as LAP. Left ventricular pressure may include systolic pressure, diastolic pressure, or end-diastolic pressure. Left ventricular pressure is also abbreviated as LVP. Left ventricular end-diastolic pressure is also abbreviated as LVEDP. Central venous pressure is also abbreviated as CVP. Pulmonary wedge pressure is also abbreviated as PWP. Pulmonary arterial wedge pressure is also abbreviated as PAWP, PCWP, or PAOP. “PAWP” is an abbreviation for pulmonary arterial wedge pressure. “PCWP” is an abbreviation for pulmonary capillary wedge pressure. “PAOP” is an abbreviation for pulmonary artery occlusion pressure. The information processing device 110 is a general-purpose computer such as a PC, a server computer such as a cloud server, a dedicated computer, or a mobile device such as a mobile phone, a smartphone, or a tablet. “PC” is an abbreviation for personal computer.

Each of the measurement devices 120 is a device that provides biological information to the information processing device 110. The measurement device 120 includes at least a pulse wave sensing device, and in the present embodiment, further includes a heart sound sensing device, and preferably further includes an electrocardiographic sensing device. The measurement device 120 is installed in a residential facility 101 such as a subject's home or a nursing facility, or in a medical facility 102 such as a hospital or a clinic.

In the present embodiment, the information processing system 100 further includes one or more terminal devices 130 and one or more communication devices 140.

Each of the terminal devices 130 includes a general-purpose computer such as a PC, or a mobile device such as a mobile phone, a smartphone, or a tablet. Similarly to the measurement device 120, the terminal device 130 is installed in the residential facility 101 or the medical facility 102. Alternatively, the terminal device 130 may be owned by the subject or a medical professional.

The communication device 140 includes a router or a gateway. Similarly to the measurement device 120, each of the communication devices 140 is installed in the residential facility 101 or the medical facility 102.

In a case where the measurement device 120 is installed in the residential facility 101, biological information is transmitted to the information processing device 110 via the communication device 140 and a network 150. Alternatively, in a case where the measurement device 120 is installed in the residential facility 101, biological information may be transmitted to the terminal device 130 via a wireless LAN. “LAN” is an abbreviation for local area network. When receiving biological information from the measurement device 120, the terminal device 130 transmits the received biological information to the information processing device 110 via the communication device 140 and the network 150.

In a case where the measurement device 120 is installed in the medical facility 102, biological information is transmitted to the information processing device 110 via an in-facility network 160, the communication device 140, and the network 150. Alternatively, in a case where the measurement device 120 is installed in the medical facility 102, biological information may be transmitted to the terminal device 130 or a server device 170 via the in-facility network 160. The server device 170 is installed in the medical facility 102. When receiving biological information from the measurement device 120, the terminal device 130 or the server device 170 transmits the received biological information to the information processing device 110 via the in-facility network 160, the communication device 140, and the network 150.

The network 150 includes the Internet, at least one WAN, at least one MAN, or any combination thereof. “WAN” is an abbreviation for wide area network. “MAN” is an abbreviation for metropolitan area network. The network 150 may include at least one wireless network, at least one optical network, or any combination thereof. The wireless network is, for example, an ad hoc network, a cellular network, a wireless LAN, a satellite communication network, or a terrestrial microwave network.

An overview of the present embodiment will be described with reference to FIGS. 1 to 5.

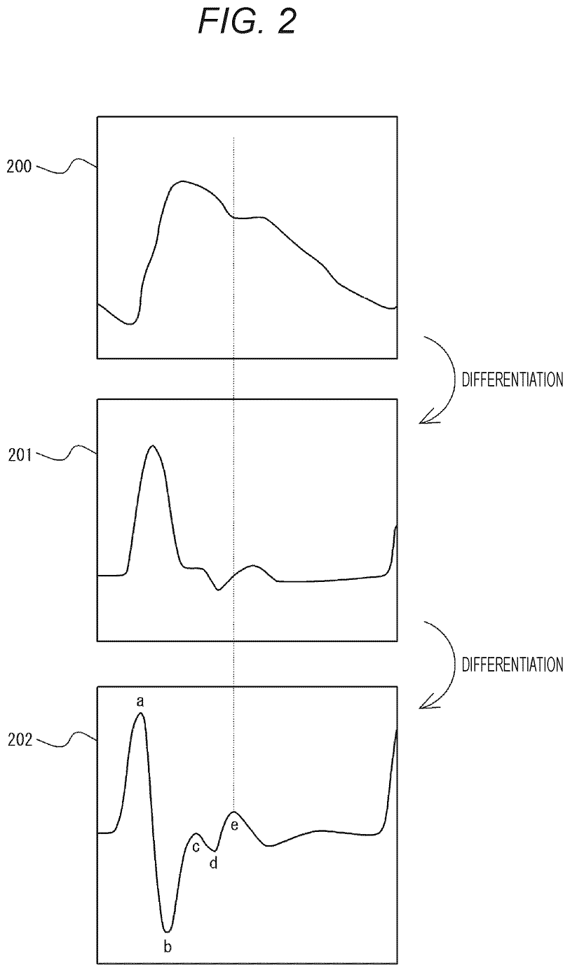

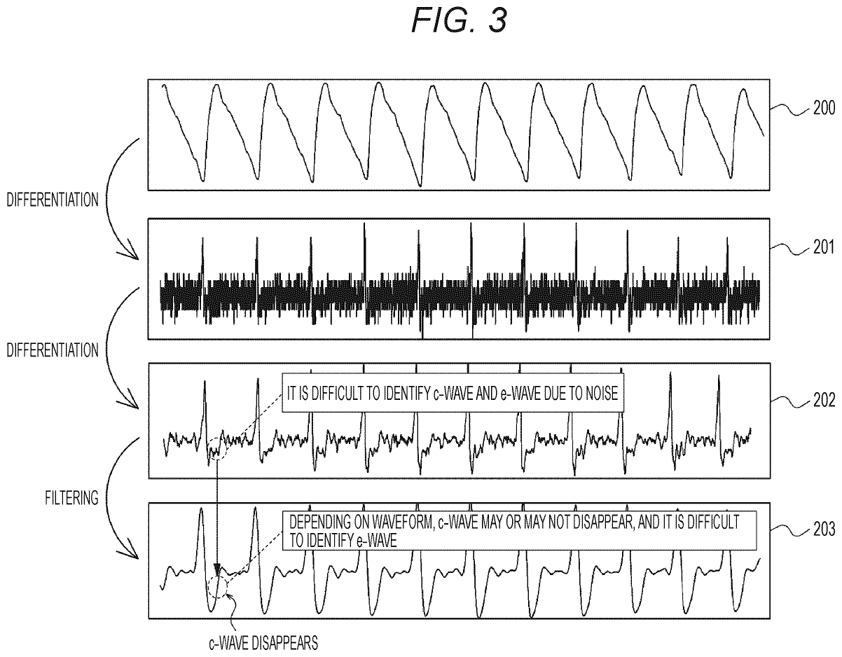

FIGS. 2 and 3 illustrate examples of a volume pulse wave waveform 200, a velocity pulse wave waveform 201, and an acceleration pulse wave waveform 202. The volume pulse wave indicates changes in blood volume flowing in a blood vessel. When the volume pulse wave is differentiated, a velocity pulse wave is obtained. The velocity pulse wave indicates changes in blood volume per unit time. When the velocity pulse wave is differentiated, an acceleration pulse wave is obtained. That is, performing second-order differentiation on the volume pulse wave yields an acceleration pulse wave. The acceleration pulse wave indicates changes in the velocity pulse wave per unit time. As illustrated in FIG. 2, the acceleration pulse wave waveform 202 includes an a-wave, a b-wave, a c-wave, a d-wave, and an e-wave for each beat. The e-wave corresponds to the dicrotic notch of the volume pulse wave necessary for estimating the intracardiac pressure. Thus, the dicrotic notch of the volume pulse wave can be identified by detecting the e-wave of the acceleration pulse wave. However, as illustrated in FIG. 3, it is difficult to identify the c-wave and the e-wave in the acceleration pulse wave waveform 202 due to noise. Thus, it is conceivable to filter the acceleration pulse wave. However, in the acceleration pulse wave waveform 202 after filtering, there are cases where the c-wave disappears and cases where the c-wave does not disappear, and it is still difficult to identify the e-wave. Thus, it is conceivable to provide a reference position corresponding to the e-wave after filtering the acceleration pulse wave.

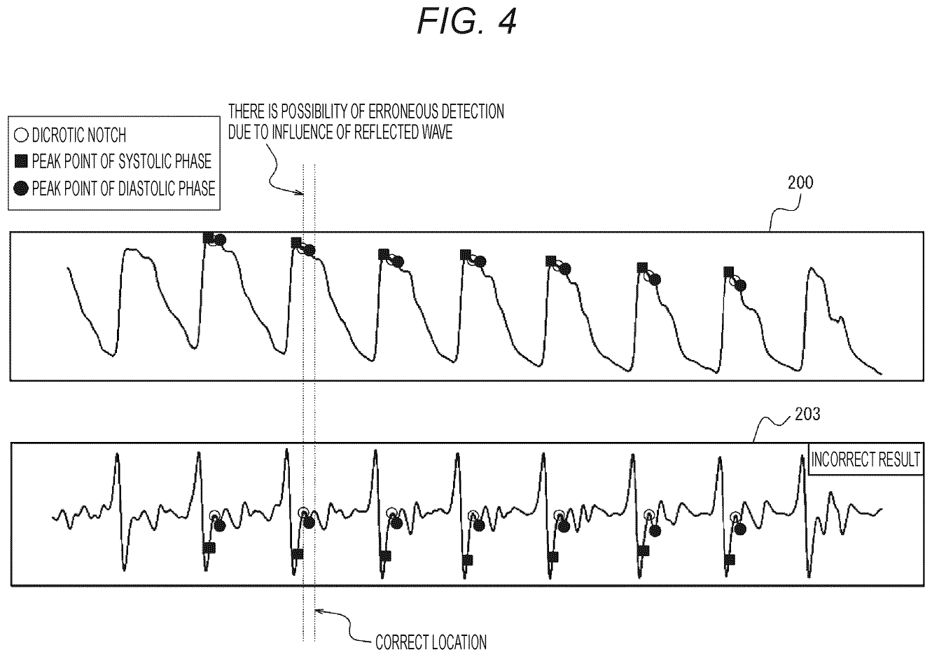

FIG. 4 illustrates an example in which a search is performed using a position corresponding to the start point of the grade II sound of the heart sound as a reference. In FIG. 4, a white circle corresponds to a dicrotic notch including an erroneous detection result. A black square corresponds to the peak point of the systolic phase. A black circle corresponds to the peak point of the diastolic phase. The systolic phase refers to the time when the ventricle is contracting, and the diastolic phase refers to the time when the ventricle is relaxing. The peak point of the systolic phase is the local maximum point of the pulse wave waveform when the ventricle is contracting, and the peak point of the diastolic phase is the local maximum point of the pulse wave waveform when the ventricle is relaxing. The dicrotic notch is a feature point that appears on the pulse wave waveform at the time of aortic valve closure, in which a rapid decrease and recovery of velocity is observed between the systolic phase and the diastolic phase. The broken line representing “there is a possibility of erroneous detection due to the influence of the reflected wave” indicates the position of the dicrotic notch erroneously detected due to using the position corresponding to the start point of the grade II sound as a reference. The broken line labelled “correct location” indicates the actual position of the dicrotic notch. In the example illustrated in FIG. 4, there is a possibility that a position different from the position corresponding to the original e-wave is erroneously detected due to the influence of reflected waves. Thus, the dicrotic notch of the volume pulse wave cannot always be correctly identified.

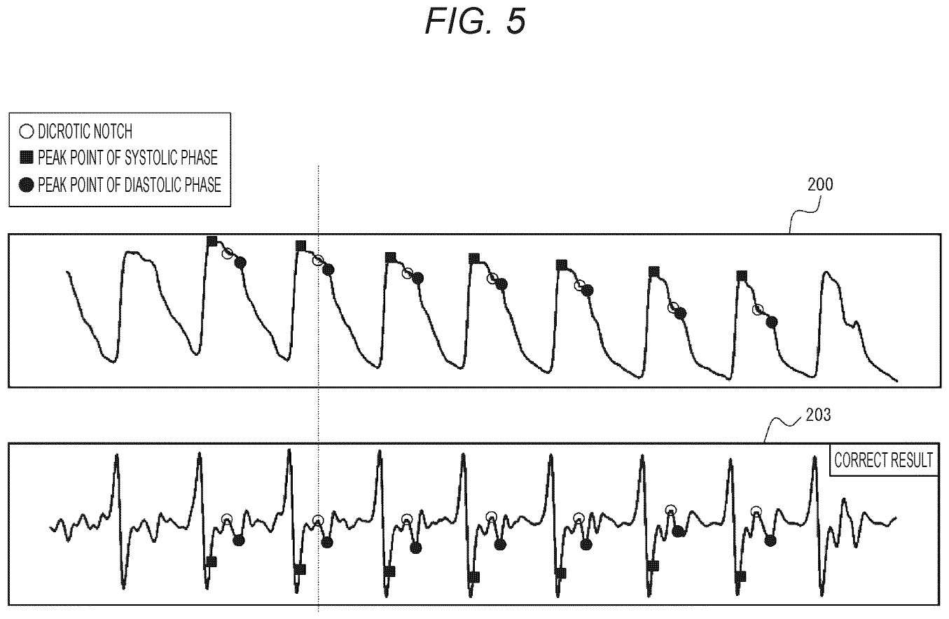

FIG. 5 illustrates an example in which a search is performed using an offset position of the acceleration pulse wave as a reference. The offset position is the position of the acceleration pulse wave after the elapse of the offset time from the position corresponding to the start point of the grade II sound of the heart sound. In the present embodiment, the offset time is a time corresponding to a time difference between the start point of the grade I sound of the heart sound and the rising point of the volume pulse wave, but may be a predetermined specified time. In FIG. 5, a white circle corresponds to the dicrotic notch. A black square corresponds to the peak point of the systolic phase. A black circle corresponds to the peak point of the diastolic phase. The broken line indicates the position of the dicrotic notch correctly detected as a result of using the offset position as a reference. In the example illustrated in FIG. 5, the accuracy of detecting the position corresponding to the e-wave can be improved. Thus, the accuracy of identifying the dicrotic notch of the volume pulse wave can also be improved.

In the present embodiment, the information processing device 110 acquires biological information. The information processing device 110 derives an acceleration pulse wave by performing second-order differentiation on the pulse wave included in the acquired biological information. As in the example illustrated in FIG. 5, the information processing device 110 identifies the dicrotic notch of the pulse wave included in the biological information by detecting a feature point corresponding to the dicrotic notch from a search range determined by an offset time of the derived acceleration pulse wave and a position corresponding to the start point of the grade II sound included in the heart sound. Therefore, according to the present embodiment, the accuracy of detecting the dicrotic notch of the pulse wave is improved.

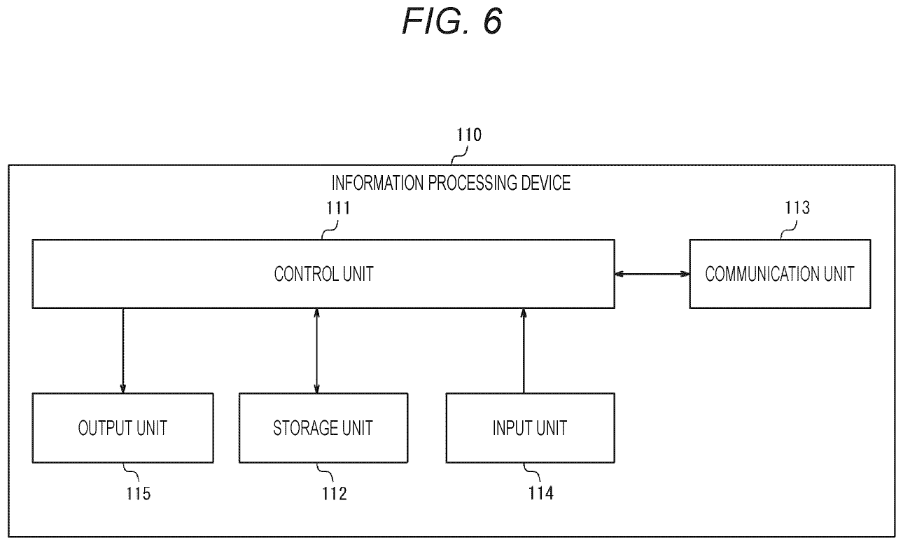

The configuration of the information processing device 110 according to the present embodiment will be described with reference to FIG. 6.

The information processing device 110 includes a control unit 111, a storage unit 112, a communication unit 113, an input unit 114, and an output unit 115.

The control unit 111 includes at least one processor, at least one programmable circuit, at least one dedicated circuit, or any combination thereof. The processor is a general-purpose processor such as a CPU or a GPU, or a dedicated processor specialized for specific processing. “CPU” is an abbreviation for central processing unit. “GPU” is an abbreviation for graphics processing unit. The programmable circuit is, for example, an FPGA. “FPGA” is an abbreviation for field-programmable gate array. The dedicated circuit is, for example, an ASIC. “ASIC” is an abbreviation for application specific integrated circuit. The control unit 111 executes processing related to the operation of the information processing device 110 while controlling each unit of the information processing device 110.

The storage unit 112 includes at least one semiconductor memory, at least one magnetic memory, at least one optical memory, or any combination thereof. The semiconductor memory is, for example, a RAM, a ROM, or a flash memory. “RAM” is an abbreviation for random access memory. “ROM” is an abbreviation for read only memory. The RAM is, for example, an SRAM or a DRAM. “SRAM” is an abbreviation for static random access memory. “DRAM” is an abbreviation for dynamic random access memory. The ROM is, for example, an EEPROM. “EEPROM” is an abbreviation for electrically erasable programmable read only memory. The flash memory is, for example, an SSD. “SSD” is an abbreviation for solid-state drive. The magnetic memory is, for example, an HDD. “HDD” is an abbreviation for hard disk drive. The storage unit 112 functions as, for example, a main storage device, an auxiliary storage device, or a cache memory. The storage unit 112 stores information used for the operation of the information processing device 110 and information obtained by the operation of the information processing device 110.

The communication unit 113 includes at least one communication module. The communication module is a module compatible with, for example, a wired LAN communication standard such as Ethernet (registered trademark), a wireless LAN communication standard such as IEEE 802.11, or a mobile communication standard such as LTE, 4G, or 5G. “IEEE” is an abbreviation for Institute of Electrical and Electronics Engineers. “LTE” is an abbreviation for Long Term Evolution. “4G” is an abbreviation for 4th generation. “5G” is an abbreviation for 5th generation. The communication unit 113 communicates with the communication device 140 via the network 150. The communication unit 113 receives information to be used for the operation of the information processing device 110 and transmits information obtained by the operation of the information processing device 110.

The input unit 114 includes at least one input device. The input device is, for example, a physical key, a capacitive key, a pointing device, a touch screen integrated with a display, a camera, or a microphone. The input unit 114 receives user operations for inputting information used for the operation of the information processing device 110. The input unit 114 may be connected to the information processing device 110 as an external input device instead of being provided in the information processing device 110. As a connection interface, an interface compatible with standards such as USB, HDMI (registered trademark), or Bluetooth (registered trademark) can be used. “USB” is an abbreviation for Universal Serial Bus. “HDMI (registered trademark)” is an abbreviation for High-Definition Multimedia Interface.

The output unit 115 includes at least one output device. The output device is, for example, a display, a printer, or a speaker. The display is, for example, an LCD or an organic EL display. “LCD” is an abbreviation for liquid crystal display. “EL” is an abbreviation for electro luminescent. The output unit 115 outputs information obtained by the operation of the information processing device 110. The output unit 115 may be connected to the information processing device 110 as an external output device instead of being provided in the information processing device 110. As the connection interface, an interface compatible with standards such as USB, HDMI (registered trademark), or Bluetooth (registered trademark) can be used.

The functions of the information processing device 110 are achieved by the processor serving as the control unit 111 executing a program. That is, the functions of the information processing device 110 are implemented by software. The program causes a computer to execute the operations of the information processing device 110 to cause the computer to function as the information processing device 110. That is, the computer functions as the information processing device 110 by executing the operations of the information processing device 110 in accordance with the program.

The program can be stored in a non-transitory computer-readable medium. The non-transitory computer-readable medium is, for example, a flash memory, a magnetic recording device, an optical disc, a magneto-optical recording medium, or a ROM. Distribution of the program is carried out, for example, by selling, transferring, or lending a portable medium such as an SD card, a DVD, or a CD-ROM that stores the program. “SD” is an abbreviation for Secure Digital. “DVD” is an abbreviation for digital versatile disc. “CD-ROM” is an abbreviation for compact disc read only memory. The program may be distributed by storing the program in a storage of a server and transferring the program from the server to another computer. The program may be provided as a program product.

The computer loads and temporarily stores, for example, the program stored in the portable medium or the program transferred from the server in the main storage device. The computer causes the processor to read the program stored in the main storage device and execute processing in accordance with the read program. The computer may read the program directly from the portable medium and execute the processing in accordance with the program. Each time the program is transferred from the server to the computer, the computer may sequentially execute processing in accordance with the received program. The processing may be executed by a so-called ASP-type service that does not transfer the program from the server to the computer, but implements the function solely through execution instructions and result acquisition. “ASP” is an abbreviation for application service provider. The program includes information intended for use in processing by an electronic computer and equivalent to the program. For example, data that is not a direct command to the computer but has a property that defines processing of the computer corresponds to the “information equivalent to the program”.

Some or all of the functions of the information processing device 110 may be implemented by a programmable circuit or a dedicated circuit as the control unit 111. That is, some or all of the functions of the information processing device 110 may be implemented by hardware.

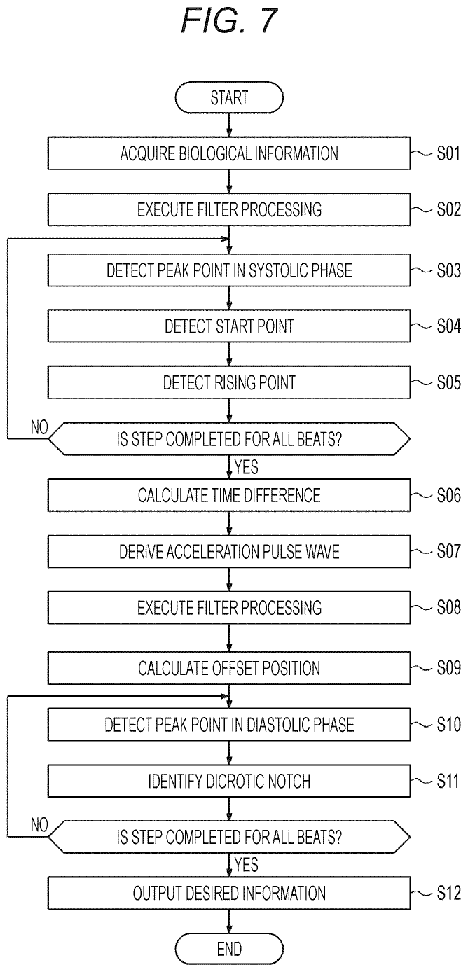

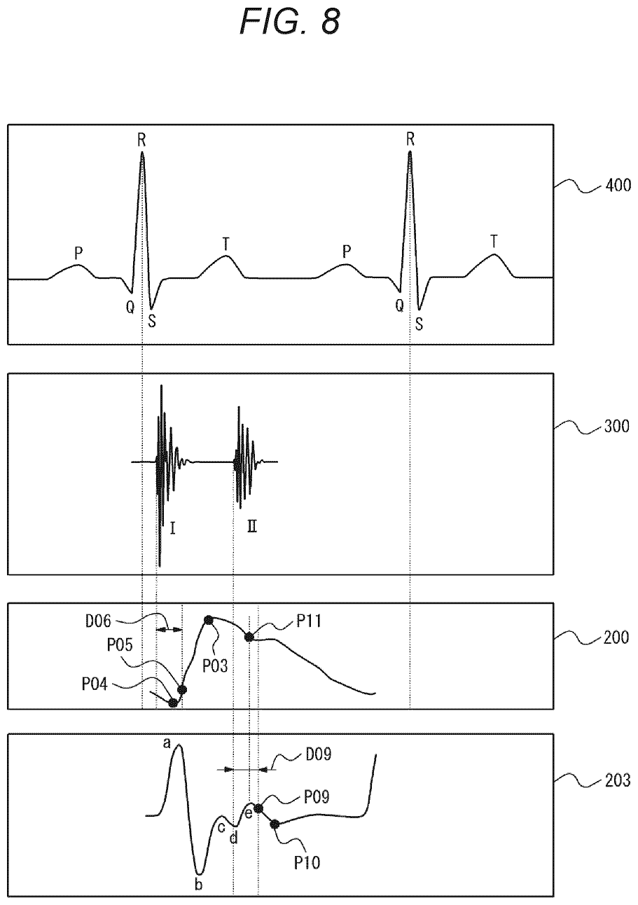

The operation of the information processing device 110 according to the present embodiment will be described with reference to FIG. 7 and FIG. 8. The operation to be described below corresponds to the information processing method according to the present embodiment. That is, the information processing method according to the present disclosure includes, for example, steps S01 to S12 illustrated in FIG. 7. FIG. 8 illustrates examples of a heart sound waveform 300 and an electrocardiogram waveform 400 that are time-synchronized with the volume pulse wave signal, in addition to the examples of the volume pulse wave waveform 200 and the acceleration pulse wave waveform 203 similar to those illustrated in FIGS. 2 to 5.

In S01, the control unit 111 acquires biological information. Specifically, the control unit 111 receives, as biological information, the measurement result including the volume pulse wave, the heart sound, and the electrocardiogram of the subject, which is obtained by the measurement device 120 via the communication unit 113. The control unit 111 stores the received biological information in the storage unit 112 and appropriately reads the biological information in steps after S02.

In S02, the control unit 111 executes filter processing on the volume pulse wave included in the biological information acquired in S01. Specifically, the control unit 111 executes noise reduction processing on the volume pulse wave in a frequency band having features such as a peak point P03 and a rising point P05, which will be described later, such as from 0.7 Hz to 7 Hz. For example, the control unit 111 applies an LPF of 10 Hz to the volume pulse wave. “LPF” is an abbreviation for low-pass filter. The filter may be a moving average filter, an FIR filter, or an IIR filter. “FIR” is an abbreviation for finite impulse response. “IIR” is an abbreviation for infinite impulse response.

The sequence of steps S03 to S05 is executed for each beat.

In S03, the control unit 111 detects the peak point P03 in the systolic phase of the volume pulse wave filtered in S02. Specifically, the control unit 111 detects a local maximum point of the volume pulse wave within a range from a position corresponding to the R-wave of the electrocardiogram included in the biological information acquired in S01 to a position corresponding to the R-wave of the next beat as the peak point P03 in the systolic phase of the volume pulse wave.

In S04, the control unit 111 detects the start point P04 of the volume pulse wave filtered in S02. Specifically, the control unit 111 detects, as the start point P04 of the volume pulse wave, the local minimum point of the volume pulse wave within a range based on the position corresponding to the R-wave of the electrocardiogram included in the biological information acquired in S01. More specifically, the control unit 111 detects, as the start point P04 of the volume pulse wave, the local minimum point of the volume pulse wave within a range from the peak point P03 in the systolic phase of the volume pulse wave detected in S03 to the position corresponding to the R-wave.

In S05, the control unit 111 detects the rising point P05 of the volume pulse wave filtered in S02. Specifically, the control unit 111 detects, as the rising point P05 of the volume pulse wave, a point that rises to a certain extent from the start point P04 of the volume pulse wave detected in S04 toward the peak point P03 in the systolic phase of the volume pulse wave detected in S03. For example, the control unit 111 detects, as the rising point P05 of the volume pulse wave, a point that rises from the start point P04 toward the peak point P03 in the systolic phase and exceeds 1/10 in the volume pulse wave.

In the present embodiment, when steps S03 to S05 are completed for all beats, steps S06 and subsequent steps are executed. In a modification example of the present embodiment, steps S06 and subsequent steps may be executed for each beat in a state where steps S03 to S05 are not completed for all beats.

In S06, the control unit 111 calculates a time difference D06 between the start point of the grade I sound included in the heart sound of the biological information acquired in S01 and the rising point P05 of the volume pulse wave detected in S05. The time difference D06 corresponds to the time difference between the pulse wave and the heart sound.

In S07, the control unit 111 derives an acceleration pulse wave by performing second-order differentiation on the volume pulse wave filtered in S02. The control unit 111 may first derive a velocity pulse wave by performing first-order differentiation on the volume pulse wave, execute filter processing on the derived velocity pulse wave, and then derive the acceleration pulse wave by performing first-order differentiation on the filtered velocity pulse wave. The filter may be a moving average filter, an FIR filter, or an IIR filter.

In S08, the control unit 111 executes filter processing on the acceleration pulse wave derived in S07. Specifically, the control unit 111 executes the noise reduction processing on the acceleration pulse wave in a frequency band having features, such as from 0.7 Hz to 7 Hz. For example, the control unit 111 applies an LPF of 10 Hz to the acceleration pulse wave. As the type of filter, a moving average filter, an FIR filter, or an IIR filter may be used.

In S09, the control unit 111 calculates an offset position P09 by adding a time D09 corresponding to the time difference D06 calculated in S06 to the position corresponding to the start point of the grade II sound included in the heart sound of the biological information acquired in S01 in the acceleration pulse wave filtered in S08.

The sequence of steps S10 to S11 is executed for each beat.

In S10, the control unit 111 detects a local minimum point P10 of the acceleration pulse wave filtered in S08, which corresponds to the peak point in the diastolic phase of the volume pulse wave. Specifically, the control unit 111 detects the first local minimum point of the acceleration pulse wave, which is subsequent to the offset position P09 calculated in S09 as the local minimum point P10 corresponding to the peak point in the diastolic phase of the volume pulse wave.

In S11, the control unit 111 identifies a dicrotic notch P11 of the volume pulse wave by detecting the feature point e corresponding to the dicrotic notch P11 of the volume pulse wave from the search range determined by the time D09 corresponding to the time difference D06 calculated in S06 and the position corresponding to the start point of the grade II sound included in the heart sound of the biological information acquired in S01 in the acceleration pulse wave filtered in S08. That is, the control unit 111 identifies the dicrotic notch P11 of the volume pulse wave by detecting the e-wave of the acceleration pulse wave. Specifically, the control unit 111 detects the feature point e using a range based on the local minimum point P10 detected in S10 as a search range. More specifically, the control unit 111 detects, as the feature point e, the first local maximum point of the acceleration pulse wave, which temporally precedes the local minimum point P10 detected in S10. That is, the control unit 111 detects, as the feature point e, the first local maximum point of the acceleration pulse wave found by backtracking along the trajectory of the acceleration pulse wave waveform 203 from the local minimum point P10 detected in S10.

For example, in a case where the offset position P09 calculated in S09 is located temporally before the dicrotic notch P11 of the volume pulse wave, that is, in a case where it precedes the dicrotic notch P11, when step S10 is skipped and step S11 is executed, there is a possibility that a feature point c is erroneously detected as the dicrotic notch. After detecting the local minimum point P10 corresponding to the peak point in the diastolic phase of the volume pulse wave, the control unit 111 detects the first local maximum point temporally preceding the local minimum point P10 as the feature point e. Therefore, the feature point e can be accurately detected even in a case where the offset position P09 calculated in S09 is located temporally before the dicrotic notch P11 of the volume pulse wave. As a result, the accuracy of identifying the dicrotic notch P11 of the volume pulse wave is further improved.

When steps S09 to S11 are completed for all beats, step S12 is executed.

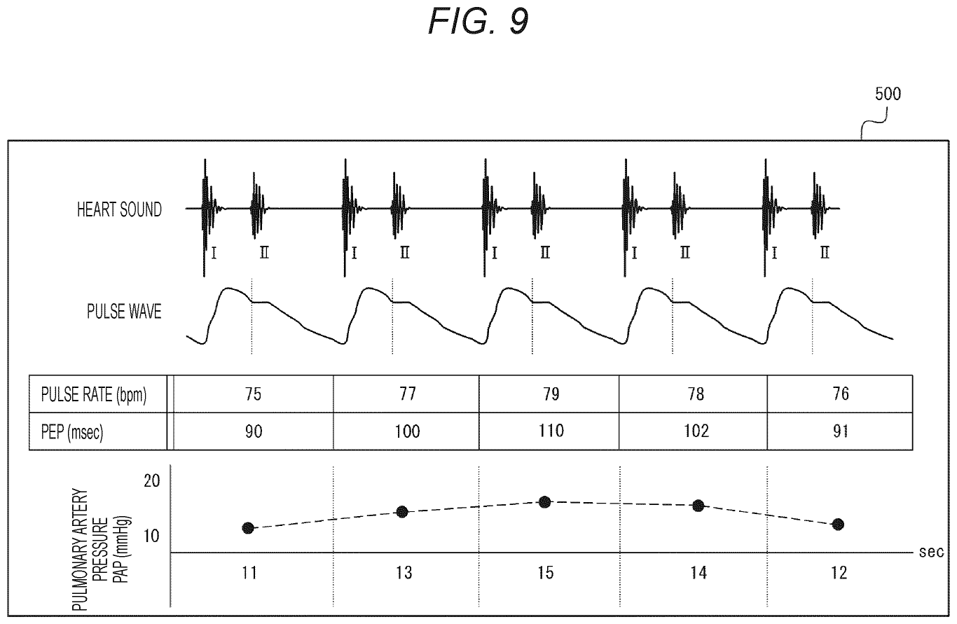

In S12, the control unit 111 outputs desired medical information including information obtained in S01 to S11, information further obtained based on the information, or both of them. As effects of displaying the information obtained in S01 to S11, for example, it is easy to grasp whether the dicrotic notch is appropriately detected, and a feature point or a temporal index such as a temporal difference calculated from the feature point can be used to determine the subject's cardiac function. The control unit 111 may estimate the intracardiac pressure such as LVEDP based on the information obtained in S01 to S11, and include the estimated intracardiac pressure in the desired information. Specifically, the control unit 111 displays a screen 500 including the desired information as illustrated in FIG. 9 on the display as the output unit 115. Alternatively, the control unit 111 may transmit the desired information to the communication device 140 via the communication unit 113. When receiving the desired information from the information processing device 110, the communication device 140 transfers the received desired information to the terminal device 130. When receiving the desired information from the communication device 140, the terminal device 130 displays the screen 500 including the received desired information on the display. The desired information includes, for example, a pulse rate, PEP, PAP, or any combination thereof in addition to the heart sound, the pulse wave, and the dicrotic notch P11 identified in S11. “PEP” is an abbreviation for pre-ejection period. The desired information may include ET, PEP/ET, or LVEDP. “ET” is an abbreviation of ejection time. By displaying information regarding the cardiac function (or cardiac function indicators), such as PEP or ET, it is possible to predict exacerbation of ischemic heart disease or heart failure. The estimated value of the intracardiac pressure such as PAP or LVEDP can be used as an index for medication prescription such as a diuretic in the cardiac disease management. Based on the estimated intracardiac pressure and patient background such as renal disease, the dosage of a recommended medication, such as a diuretic, may be calculated and displayed to individually present the optimal prescription. The trend of the estimated intracardiac pressure may be displayed, and the risk score for death or readmission at the time of discharge at the present time may be calculated and displayed from the rate of decrease from the base value measured at the time of emergency and the patient background such as the number of readmissions to present the optimal discharge timing. Such presentation may lead to prevention of readmission.

In the present embodiment, the time D09 corresponding to the time difference D06 between the start point of the grade I sound and the rising point P05 of the volume pulse wave, which corresponds to the a-wave that is less likely to be affected by noise is applied as an offset between the start point of the grade II sound and the e-wave that is likely to be affected by noise. Therefore, detection in consideration of individual differences becomes possible and the accuracy of detecting the e-wave can be improved. As a result, the accuracy of detecting the dicrotic notch P11 of the volume pulse wave is improved.

According to a modification example of the present embodiment, instead of the time D09 corresponding to the time difference D06 between the start point of the grade I sound and the rising point P05 of the volume pulse wave, the time corresponding to the time difference between the start point of the grade I sound and the a-wave may be applied as the offset between the start point of the grade II sound and the e-wave.

According to another modification example of the present embodiment, a predetermined specified time may be applied as the offset between the start point of the grade II sound and the e-wave without calculating the time difference. In such a modification example, the control unit 111 calculates the offset position by adding a predetermined specified time to the position of the acceleration pulse wave, which corresponds to the start point of the grade II sound included in the heart sound, instead of the time D09. Similarly to step S10, the control unit 111 detects the first local minimum point after the calculated offset position as the local minimum point P10 corresponding to the peak point in the diastolic phase of the volume pulse wave. Similarly to step S11, the control unit 111 detects the feature point e corresponding to the dicrotic notch P11 of the volume pulse wave from the range of the acceleration pulse wave based on the detected local minimum point P10, and thus identifies the dicrotic notch P11 of the volume pulse wave. More specifically, the control unit 111 detects, as the feature point e, the first local maximum point of the acceleration pulse wave, which precedes the detected local minimum point P10. As effects of using the specified time as in this modification example, for example, it is possible to save time and effort in calculating the time difference and to accurately detect the notch since the search range can be limited.

According to still another modification example of the present embodiment, a higher-order pulse wave may be used instead of the second-order differentiated acceleration pulse wave.

The present disclosure is not limited to the above-described embodiments. For example, two or more blocks illustrated in the block diagram may be integrated, or one block may be divided. Instead of executing two or more steps described in the flowchart in time series according to the description, the steps may be executed in parallel or in a different order, depending on the processing capability of the device that executes each step or as necessary. In addition, modifications can be made without departing from the gist of the present disclosure.

What is claimed is:

1. A biological information processing device comprising:
an interface connectable to a measurement device for measuring biological information including a volume pulse wave and a heart sound;
a memory; and
a processor configured to execute a program stored in the memory to:
acquire the biological information,
perform second-order differentiation on the volume pulse wave included in the biological information and derive an acceleration pulse wave from the volume pulse wave,
determine, in the acceleration pulse wave, an offset position occurring an offset time after the beginning of a grade II sound included in the heart sound,
backtrack from a local minimum point that is subsequent to the offset position to find a first local maximum point,
determine a point in the volume pulse wave corresponding to the first local maximum point to be a dicrotic notch of the volume pulse wave, and
store information indicating the dicrotic notch in association with or as part of the biological information.

2. The biological information processing device according to claim 1, wherein
the local minimum point is a first local minimum point after the offset position and before the beginning of a next pulse.

3. The biological information processing device according to claim 1, wherein
the processor executes the program to determine, as the offset time, a time difference between a start point of a grade I sound included in the heart sound and a rising point of the volume pulse wave.

4. The biological information processing device according to claim 3, wherein
the biological information further includes an electrocardiogram, and
the processor executes the program to:
determine, as a start point, a local minimum point of the volume pulse wave within a range between an R-wave shown in the electrocardiogram and a systolic peak point of the volume pulse wave, and
determine, as the rising point, a point at which the volume pulse wave has risen from the start point toward the systolic peak point by a predetermined extent.

5. The biological information processing device according to claim 1, wherein
the offset time is a predetermined specified time stored in the memory.

6. The biological information processing device according to claim 1, wherein
the processor executes the program to execute filter processing on the acceleration pulse wave before determining the offset position.

7. The biological information processing device according to claim 1, wherein
the processor executes the program to determine, based on the determined dicrotic notch, at least one intracardiac pressure selected from a group consisting of: pulmonary artery pressure, left ventricular end-diastolic pressure, and pulmonary arterial wedge pressure.

8. The biological information processing device according to claim 7, wherein
the processor executes the program to:
acquire patient background information including a presence of renal disease, and
calculate and output a recommended dosage of a diuretic medication based on a combination of the determined intracardiac pressure and the acquired patient background information.

9. The biological information processing device according to claim 7, wherein
the processor is further configured to:
monitor a trend in the intracardiac pressure of a hospitalized patient over time,
calculate a risk score for readmission or death based on a rate of decrease in the intracardiac pressure from a base value measured during an emergency, and
determine a discharge timing for the patient based on the calculated risk score.

10. The biological information processing device according to claim 1, wherein
the processor executes the program to determine, based on the determined dicrotic notch, a cardiac function indicator that includes at least one of:
ejection time calculated as a time difference between a rising point of the volume pulse wave and the dicrotic notch, and
a ratio of pre-ejection period to the ejection time.

11. A biological information processing method comprising:
acquiring biological information including a volume pulse wave and a heart sound;
deriving an acceleration pulse wave by performing second-order differentiation on the volume pulse wave included in the biological information;
determining, in the acceleration pulse wave, an offset position occurring an offset time after the beginning of a grade II sound included in the heart sound;
backtracking from a local minimum point that is subsequent to the offset position to find a first local maximum point;
determining a point in the volume pulse wave corresponding to the first local maximum point to be a dicrotic notch of the volume pulse wave; and
storing information indicating the dicrotic notch in association with or as part of the biological information.

12. The biological information processing method according to claim 11, wherein
the local minimum point is a first local minimum point after the offset position and before the beginning of a next pulse.

13. The biological information processing method according to claim 11, further comprising:
determining, as the offset time, a time difference between a start point of a grade I sound included in the heart sound and a rising point of the volume pulse wave.

14. The biological information processing method according to claim 13, wherein
the biological information further includes an electrocardiogram, and
the method further comprises:
determining, as a start point, a local minimum point of the volume pulse wave within a range between an R-wave shown in the electrocardiogram and a systolic peak point of the volume pulse wave; and
determining, as the rising point, a point at which the volume pulse wave has risen from the start point toward the systolic peak point by a predetermined extent.

15. The biological information processing method according to claim 11, wherein
the offset time is a predetermined specified time retrieved from a memory.

16. The biological information processing method according to claim 11, further comprising:
executing filter processing on the acceleration pulse wave before determining the offset position.

17. The biological information processing method according to claim 11, further comprising:
determining, based on the determined dicrotic notch, at least one intracardiac pressure selected from a group consisting of: pulmonary artery pressure, left ventricular end-diastolic pressure, and pulmonary arterial wedge pressure.

18. The biological information processing method according to claim 17, further comprising:
acquiring patient background information including a presence of renal disease; and
calculating and outputting a recommended dosage of a diuretic medication based on a combination of the determined intracardiac pressure and the acquired patient background information.

19. The biological information processing method according to claim 17, further comprising:
monitoring a trend in the intracardiac pressure of a hospitalized patient over time;
calculating a risk score for readmission or death based on a rate of decrease in the intracardiac pressure from a base value measured during an emergency; and
determine a discharge timing for the patient based on the calculated risk score.

20. A non-transitory computer-readable medium storing a program that causes a computer to execute biological information processing comprising:
acquiring biological information including a volume pulse wave and a heart sound;
deriving an acceleration pulse wave by performing second-order differentiation on the volume pulse wave included in the biological information;
determining, in the acceleration pulse wave, an offset position occurring an offset time after the beginning of a grade II sound included in the heart sound;
backtracking from a local minimum point that is subsequent to the offset position to find a first local maximum point;
determining a point in the volume pulse wave corresponding to the first local maximum point to be a dicrotic notch of the volume pulse wave; and
storing information indicating the dicrotic notch in association with or as part of the biological information.
